(19) United States
(12) Patent Application Publication
(10) Pub. No.: US 2026/0263146 A1
(43) Pub. Date: Sep. 10, 2026

(54) VASCULAR ACCESS CLOSURE SYSTEMS AND METHODS

(71) Applicant: Boston Scientific Scimed, Inc., Maple Grove, MN (US)

(72) Inventors: Brady Scott Logan, Monticello, MN (US); Jaydeep Y. Kokate, Minneapolis, MN (US); Derek Kenneth Larson, Golden Valley, MN (US); Andrew Jonathan Miller, Phoenixville, PA (US); Michael Dotsey, Chester Springs, PA (US)

(73) Assignee: Boston Scientific Scimed, Inc., Maple Grove, MN (US)

(21) Appl. No.: 19/558,147

(22) Filed: Mar. 5, 2026

Related U.S. Application Data
(60) Provisional application No. 63/767,161, filed on Mar. 5, 2025.

(51) Int. Cl. A61B 18/14; A61B 18/00

(52) U.S. Cl. CPC A61B 18/148; A61B 2018/00214; A61B 2018/00404; A61B 2018/0063

(57) ABSTRACT

A closure system for sealing an access site in a blood vessel includes a device handle, a catheter assembly, an expandable member coupled to the catheter assembly, and a sealing shaft assembly connected to a source of RF energy. The sealing shaft assembly includes a second shaft portion movably coupled to a first shaft portion. Distal ends of the first and second shaft portions are axially offset in an open configuration. The catheter assembly is disposed between the first and second shaft portions. A method of sealing the access site includes advancing the catheter assembly into the vessel, shifting the expandable member to the deployed configuration, retracting the closure system to engage the expandable member with the vessel, exposing the sealing shaft assembly, actuating the sealing shaft assembly to grasp the vessel, and applying RF energy to the vessel.

### CROSS-REFERENCE TO RELATED APPLICATIONS

### This application claims the benefit of priority under 35 U.S.C. §119 of U.S. Provisional Application No. 63/767,161, filed Mar. 5, 2025, the entire disclosure of which is hereby incorporated by reference.

### TECHNICAL FIELD

The disclosure relates generally to medical devices and more particularly to systems and methods for closing vascular access sites.

### BACKGROUND

A wide variety of intracorporeal medical devices have been developed for medical use, and more specifically for surgical and/or intravascular use. Some existing mechanisms for closing vascular access sites use sealants, sutures, clips, staples, or other mechanical closure structures left behind at the access site. These devices are manufactured by any one of a variety of different manufacturing methods and may be used according to any one of a variety of methods. There is an ongoing need to provide alternative medical devices as well as alternative methods for manufacturing and/or using medical devices.

### SUMMARY

In one example, a closure system for sealing an access site through a side wall of a blood vessel of a patient may comprise a device handle, a catheter assembly extending distally from the device handle, an expandable member coupled to a distal end of the catheter assembly and configured to shift between a delivery configuration and a deployed configuration, wherein the expandable member may be configured to engage an interior surface of the side wall of the blood vessel in the deployed configuration, and a sealing shaft assembly extending distally from the device handle and operatively connected to a source of RF energy, wherein the sealing shaft assembly may be configured to apply RF energy to the side wall of the blood vessel at the access site.

In addition, or alternatively, to any example described herein, the expandable member comprises a framework including a plurality of tissue-engaging elements extending therefrom.

In addition, or alternatively, to any example described herein, the expandable member is self-biased toward the deployed configuration.

In addition, or alternatively, to any example described herein, the catheter assembly comprises an outer tubular member fixedly secured to a first end of the expandable member and an inner shaft member fixedly secured to a second end of the expandable member.

In addition, or alternatively, to any example described herein, the inner shaft member is axially movable relative to the outer tubular member to shift the expandable member between the delivery configuration and the deployed configuration.

In addition, or alternatively, to any example described herein, the catheter assembly extends within the sealing shaft assembly.

In addition, or alternatively, to any example described herein, the sealing shaft assembly comprises a first shaft portion fixedly secured to the device handle and a second shaft portion movably coupled to the first shaft portion, wherein the second shaft portion is operatively coupled to an actuator of the device handle.

In addition, or alternatively, to any example described herein, the actuator is configured to move the second shaft portion relative to the first shaft portion to shift the sealing shaft assembly between an open configuration and a closed configuration.

In addition, or alternatively, to any example described herein, the sealing shaft assembly comprises a linkage configured to cooperate with the actuator to move the second shaft portion axially and laterally relative to the first shaft portion.

In addition, or alternatively, to any example described herein, in the closed configuration, the first shaft portion and the second shaft portion cooperate to exert a compressive force against the catheter assembly.

In addition, or alternatively, to any example described herein, the catheter assembly is axially translatable relative to the sealing shaft assembly when the sealing shaft assembly is in the closed configuration.

In addition, or alternatively, to any example described herein, the first shaft portion comprises a first polarity electrode and the second shaft portion comprises a second polarity electrode.

In addition, or alternatively, to any example described herein, and in a second example, a closure system for sealing an access site through a side wall of a blood vessel of a patient may comprise a device handle, a catheter assembly extending distally from the device handle, an expandable member coupled to a distal end of the catheter assembly and configured to shift between a delivery configuration and a deployed configuration, wherein the expandable member may be configured to engage an interior surface of the side wall of the blood vessel in the deployed configuration, and a sealing shaft assembly extending distally from the device handle and operatively connected to a source of RF energy, wherein the sealing shaft assembly may be configured to apply RF energy to the side wall of the blood vessel at the access site. The sealing shaft assembly may comprise a first shaft portion fixedly secured to the device handle and a second shaft portion movably coupled to the first shaft portion, wherein a distal end of the second shaft portion may be axially offset from a distal end of the first shaft portion in an open configuration. The catheter assembly may be slidably disposed between the first shaft portion and the second shaft portion.

In addition, or alternatively, to any example described herein, the closure system is devoid of any structure configured to be left behind within the blood vessel after the sealing shaft assembly applies RF energy to the side wall of the blood vessel.

In addition, or alternatively, to any example described herein, the closure system is devoid of any structure configured to be left attached to the blood vessel after the sealing shaft assembly applies RF energy to the side wall of the blood vessel.

In addition, or alternatively, to any example described herein, and in a third example, a method of sealing an access site through a side wall of a blood vessel may comprise: advancing a catheter assembly of a closure system over a guidewire into the blood vessel through the access site; shifting an expandable member coupled to the distal end of the catheter assembly from a delivery configuration to a deployed configuration within the blood vessel; retracting the closure system to engage the expandable member with an interior surface of the side wall of the blood vessel; exposing a sealing shaft assembly operatively connected to a source of RF energy adjacent the access site; actuating the sealing shaft assembly to grasp the side wall of the blood vessel around the catheter assembly; and applying RF energy to the side wall of the blood vessel at the access site with the sealing shaft assembly.

In addition, or alternatively, to any example described herein, the method may comprise, prior to applying RF energy to the side wall of the blood vessel, and while grasping the side wall of the blood vessel with the sealing shaft assembly, retracting the expandable member within the catheter assembly.

In addition, or alternatively, to any example described herein, the method may comprise, after applying RF energy to the side wall of the blood vessel, actuating the sealing shaft assembly to release the side wall of the blood vessel.

In addition, or alternatively, to any example described herein, the sealing shaft assembly comprises a first shaft portion fixedly secured to a device handle of the closure system and a second shaft portion movably coupled to the first shaft portion, wherein the second shaft portion is operatively coupled to an actuator of the device handle.

In addition, or alternatively, to any example described herein, the sealing shaft assembly comprises a linkage configured to cooperate with the actuator to move the second shaft portion axially and laterally relative to the first shaft portion.

The above summary of some embodiments, aspects, and/or examples is not intended to describe each disclosed embodiment or every implementation of the present disclosure. The figures and the detailed description more particularly exemplify aspects of these embodiments.

### BRIEF DESCRIPTION OF THE DRAWINGS

The disclosure may be more completely understood in consideration of the following detailed description in connection with the accompanying drawings, in which:

FIGS. 1-18 illustrate selected aspects of a closure system for and a method of sealing an access site through a side wall of a blood vessel of a patient.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale and/or which may include changes of scale therein, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently – such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The systems, devices, and/or methods disclosed herein may provide a number of desirable features and benefits as described in more detail below.

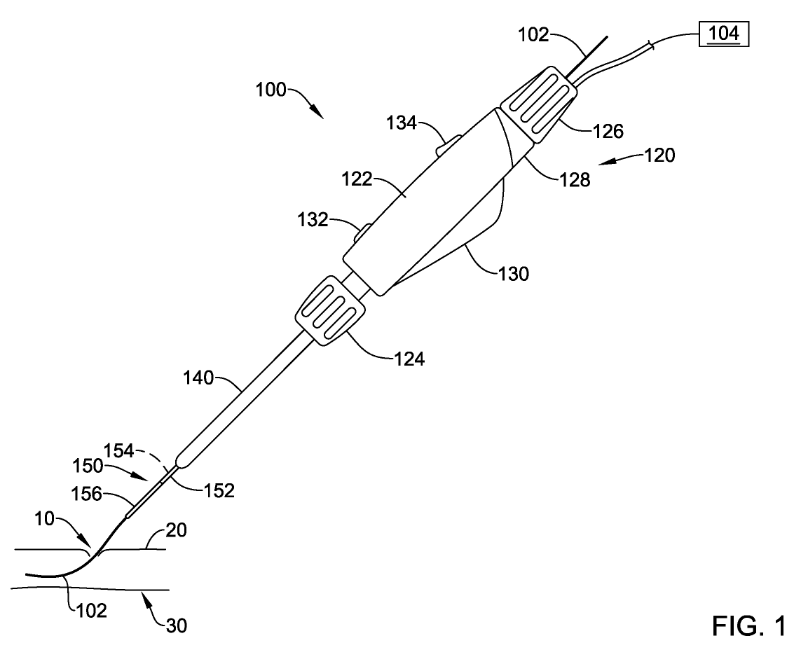

FIGS. 1-18 illustrate selected aspects of a closure system 100 (e.g., an access closure device) for sealing an access site 10 through a side wall 20 of a blood vessel 30 of a patient, and associated methods of sealing the access site 10 through the side wall 20 of the blood vessel 30 of a patient. In some embodiments, the closure system 100 may comprise a guidewire 102 and/or may be configured to be advanced over the guidewire 102 to the access site 10 and/or into the lumen of the blood vessel 30 of the patient, as seen in FIG. 1. In some embodiments, the guidewire 102 may be advanced into the lumen of the blood vessel 30 of the patient through the access site 10 and/or the side wall 20 of the blood vessel 30 to facilitate a treatment procedure performed percutaneously. In some embodiments, the access site 10 may have a diameter greater than 8 French. In some embodiments, the access site 10 may have a diameter between about 8 French and about 28 French. In some embodiments, the access site 10 may have a diameter between about 16 French and about 24 French. Other configurations and/or sizes are also contemplated. Following the treatment procedure, the access site 10 must be closed and/or sealed in order to prevent blood loss, particularly for large bore access sites (e.g., greater than 8 French). Manual compression is a commonly used closure method for small bore access sites (e.g., less than 8 French). Using an access closure device instead of manual compression for closing large bore access sites may shorten hospitalization, reduce hospital costs, and/or minimize vascular complications. In at least some embodiments, the guidewire 102 used for the treatment procedure may be used with the closure system 100 and/or the associated methods.

In some embodiments, the closure system 100 may comprise a device handle 120. In some embodiments, the device handle 120 may comprise a main handle 122 configured to be gripped by a user’s hand. In some embodiments, the device handle 120 may comprise a distal knob 124. In some embodiments, the device handle 120 may comprise a proximal knob 126. In some embodiments, the device handle 120 may comprise a proximal slide 128. In some embodiments, the device handle 120 may comprise an actuator 130. In some embodiments, the actuator 130 may comprise a lever movable relative to the main handle 122. In some embodiments, the actuator 130 may be pivotably coupled to the main handle 122. Other configurations are also contemplated. In some embodiments, the device handle 120 may comprise an RF (radio frequency) actuation button 132. In some embodiments, the device handle 120 may comprise an actuator release button 134. Additional description related to the device handle 120 and/or elements thereof, the function of and/or relationship(s) between the device handle 120 and/or elements thereof and other elements of the closure system 100, etc. is provided below. Some suitable but non-limiting materials for the device handle 120 and/or elements thereof, etc., including but not limited to polymeric materials, metallic materials, and/or composite materials, are described below. In some embodiments, the device handle 120 and/or elements thereof may preferably be formed from a polymeric material. Other configurations and/or materials are also contemplated.

In some embodiments, the closure system 100 may comprise an outer sheath 140 extending distally from the device handle 120. Some suitable but non-limiting materials for the outer sheath 140, including but not limited to polymeric materials, metallic materials, and/or composite materials, are described below. In one non-limiting example, the outer sheath 140 may preferably be formed from a polymeric material. Other configurations and/or materials are also contemplated. The outer sheath 140 may comprise a distal opening 142. In some embodiments, the distal opening 142 of the outer sheath 140 may face and/or open distally.

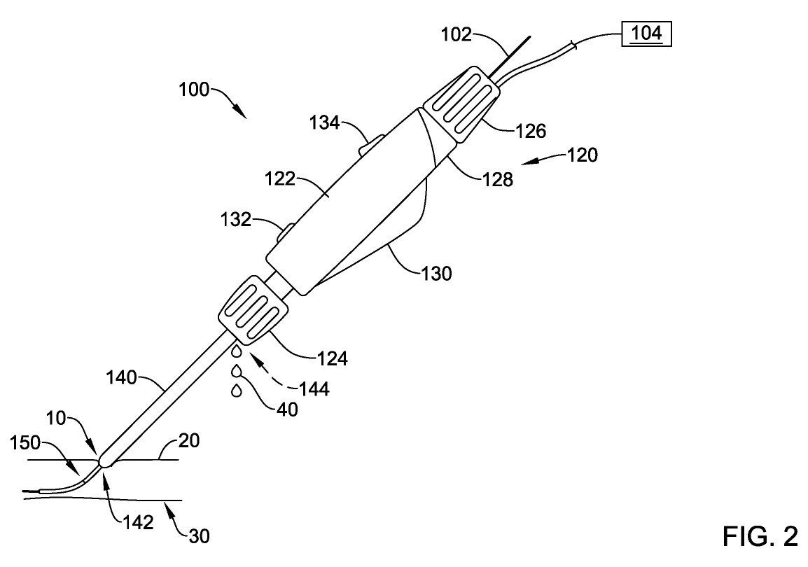

In some embodiments, the method of sealing the access site 10 through the side wall 20 of the blood vessel 30 of the patient may comprise advancing the outer sheath 140 into the access site 10 through the side wall 20 of the blood vessel 30 of the patient, as seen in FIG. 2. In some embodiments, the method of sealing the access site 10 through the side wall 20 of the blood vessel 30 of the patient may comprise verifying proper insertion depth of the closure system 100 and/or the outer sheath 140 via bleed back through a weep hole 144 at a proximal region of the outer sheath 140. For example, blood 40 from within the blood vessel 30 of the patient may travel within and/or through the outer sheath 140 and pass through the weep hole 144 when the distal opening 142 of the outer sheath 140 is inserted against and/or into the access site 10. Other configurations and/or locations for the weep hole 144 are also contemplated.

In some embodiments, the closure system 100 may comprise a catheter assembly 150 extending distally from the device handle 120. In some embodiments, the method of sealing the access site 10 through the side wall 20 of the blood vessel 30 of the patient may comprise advancing the catheter assembly 150 of the closure system 100 over and/or along the guidewire 102 into the blood vessel 30 through the access site 10 and/or the side wall 20 of the blood vessel 30, as seen in FIG. 2. In at least some embodiments, the method of sealing the access site 10 through the side wall 20 of the blood vessel 30 of the patient may comprise advancing the outer sheath 140 and the catheter assembly 150 of the closure system 100 over and/or along the guidewire 102 into the blood vessel 30 through the access site 10 and/or the side wall 20 of the blood vessel 30 simultaneously.

In some embodiments, the catheter assembly 150 may be fixedly attached to and/or secured to the proximal knob 126 and/or the proximal slide 128. In some embodiments, the catheter assembly 150 may comprise a guidewire lumen extending therethrough, wherein the guidewire lumen may be configured to slidably receive the guidewire 102 therein. In some embodiments, the catheter assembly 150 may comprise an outer tubular member 152 and an inner shaft member 154. Some suitable but non-limiting materials for the catheter assembly 150, the outer tubular member 152, and/or the inner shaft member 154, etc., including but not limited to polymeric materials, metallic materials, and/or composite materials, are described below. In some embodiments, the catheter assembly 150, the outer tubular member 152, and/or the inner shaft member 154 may preferably be formed from a polymeric material. Other configurations and/or materials are also contemplated.

In some embodiments, the outer tubular member 152 and the inner shaft member 154 may be axially movable and/or axially translatable relative to each other. In some embodiments, a proximal end of the outer tubular member 152 may be fixedly attached to and/or secured to the proximal knob 126. In some embodiments, a proximal end of the inner shaft member 154 may be fixedly attached to and/or secured to the proximal slide 128. In some embodiments, axial movement of the proximal knob 126 relative to the proximal slide 128 may be configured to axially move and/or axially translate the outer tubular member 152 relative to the inner shaft member 154.

In some embodiments, the proximal knob 126 may be configured to axially move and/or axially translate the catheter assembly 150 and/or the outer tubular member 152 relative to the device handle 120 and/or the main handle 122. In some embodiments, the proximal slide 128 may be configured to axially move and/or axially translate the catheter assembly 150 and/or the inner shaft member 154 relative to the device handle 120 and/or the main handle 122.

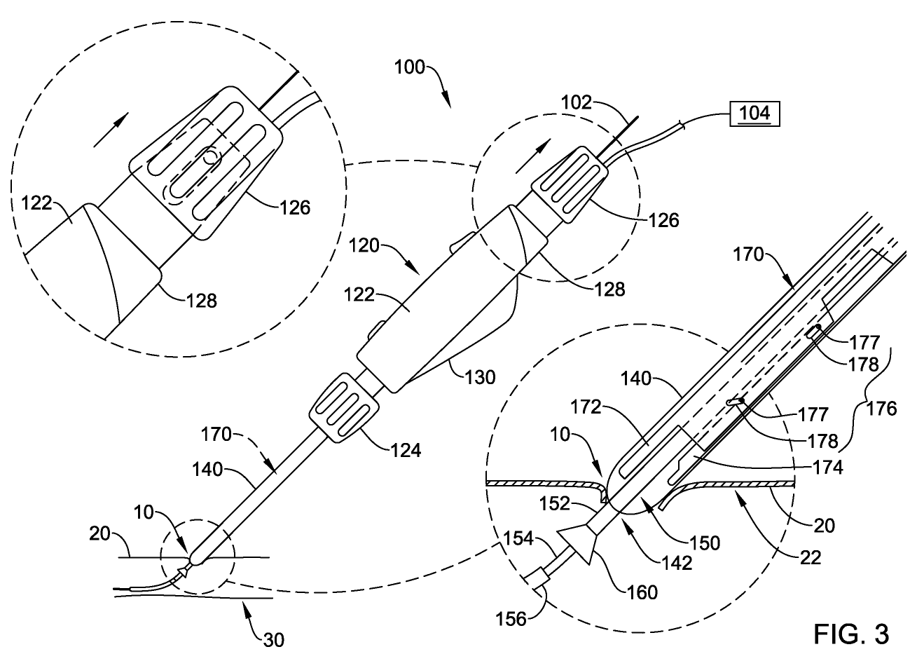

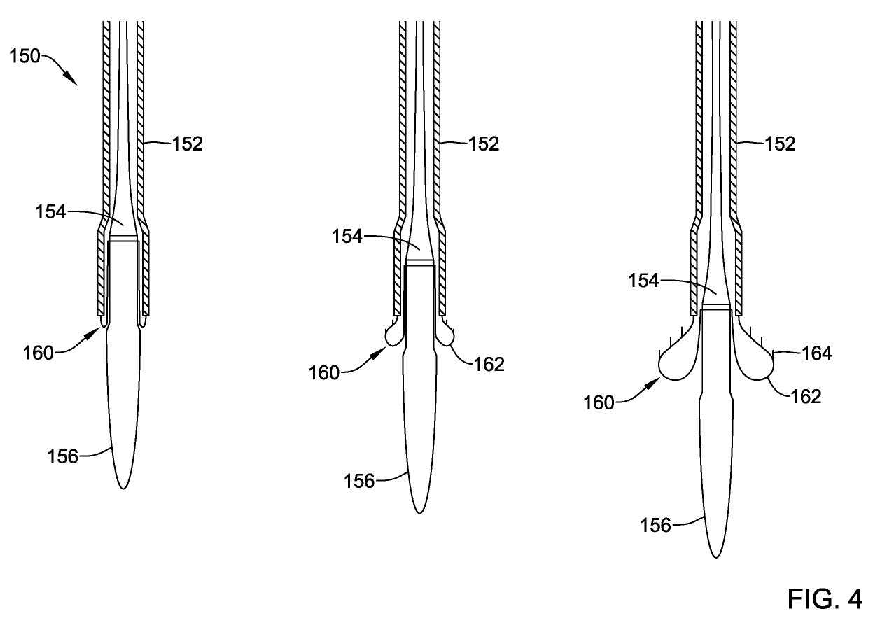

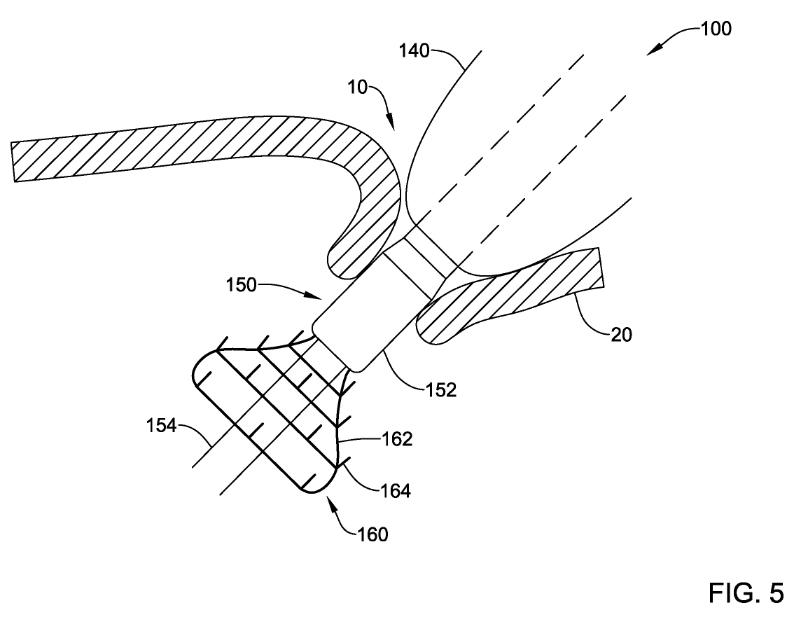

In some embodiments, the closure system 100 may comprise an expandable member 160 coupled to a distal end of the catheter assembly 150, as seen in FIG. 3. In some embodiments, the expandable member 160 may be configured to shift between a delivery configuration and a deployed configuration, as seen in partial cross-section in FIG. 4. In some embodiments, the expandable member 160 may comprise a balloon or an inflatable member. In some embodiments, the expandable member 160 may comprise a plurality of traction members configured to expand out from the catheter assembly 150 to grip adjacent tissue (e.g., the side wall 20 of the blood vessel 30). In some embodiments, the expandable member 160 may comprise a framework 162 including a plurality of tissue-engaging elements 164 extending therefrom, as seen in FIG. 5. In some embodiments, in the delivery configuration, the plurality of tissue-engaging elements 164 may be aligned with the framework 162 and/or may not project radially outward of and/or from the catheter assembly 150. In some embodiments, in the deployed configuration, the plurality of tissue-engaging elements 164 may project away from the framework 162 and/or may project radially outward of and/or from the catheter assembly 150 to engage with adjacent tissue (e.g., the side wall 20 of the blood vessel 30). In some embodiments, the expandable member 160 may be self-biased toward and/or to the deployed configuration.

In some embodiments, the expandable member 160 may be formed from a tubular member that is cut (such as via a laser or machining) with a desired pattern for the framework 162 and/or the plurality of tissue-engaging elements 164. Some suitable but non-limiting materials for the expandable member 160, etc., including but not limited to polymeric materials, metallic materials, and/or composite materials, are described below. In one non-limiting example, the expandable member 160 may preferably be formed from nickel-titanium alloy (e.g., nitinol). In another non-limiting example, the expandable member 160 may be formed from stainless steel. Other configurations and/or materials are also contemplated.

In some embodiments, the expandable member 160 may be disposed between the outer tubular member 152 and the inner shaft member 154 in the delivery configuration. In some embodiments, the expandable member 160 may extend between the outer tubular member 152 and the inner shaft member 154. In some embodiments, the outer tubular member 152 of the catheter assembly 150 may be fixedly attached and/or fixedly secured to a first end of the expandable member 160 and the inner shaft member 154 may be fixedly attached and/or fixedly secured to a second end of the expandable member 160. In some embodiments, the inner shaft member 154 may comprise a distal tip member 156 extending distal of the outer tubular member 152. In some embodiments, the second end of the expandable member 160 may be fixedly attached and/or fixedly secured to a proximal end of the distal tip member 156. In some embodiments, the proximal end of the distal tip member 156 may be disposed within the outer tubular member 152 in the delivery configuration and the proximal end of the distal tip member 156 may be spaced apart distally from a distal end of the outer tubular member 152 in the deployed configuration. In some embodiments, the outer tubular member 152 may be configured to constrain the expandable member 160 in the delivery configuration. For example, the expandable member 160 may be disposed and/or constrained within the outer tubular member 152 in the delivery configuration.

In some embodiments, the inner shaft member 154 may be axially movable and/or axially translatable relative to the outer tubular member 152 to shift the expandable member 160 between the delivery configuration and the deployed configuration, as seen in FIG. 4. In some embodiments, the outer tubular member 152 may be axially movable and/or axially translatable relative to the inner shaft member 154 to shift the expandable member 160 between the delivery configuration and the deployed configuration. In one non-limiting example, the proximal knob 126 may be configured to axially move and/or axially translate in a proximal direction to axially move and/or axially translate the outer tubular member 152 relative to the inner shaft member 154 and/or the distal tip member 156 to shift the expandable member 160 from the delivery configuration toward and/or to the deployed configuration, as seen in FIG. 3.

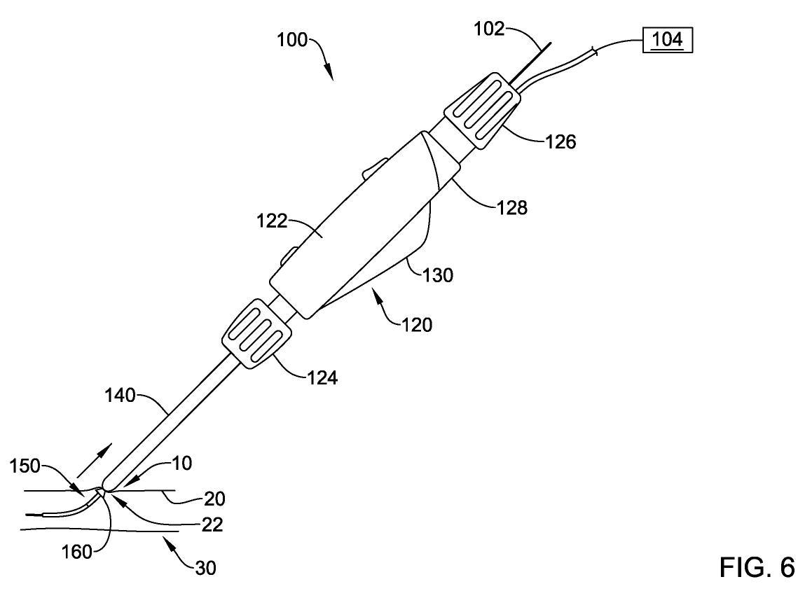

In some embodiments, the device handle 120 and/or the proximal knob 126 may be configured to shift the expandable member 160 from the delivery configuration toward and/or to the deployed configuration within the blood vessel 30 of the patient. In some embodiments, the expandable member 160 may be configured to engage an interior surface 22 of the side wall 20 of the blood vessel 30 in the deployed configuration (e.g., after being shifted to the deployed configuration within the blood vessel 30). In some embodiments, the method of sealing the access site 10 through the side wall 20 of the blood vessel 30 of the patient may comprise shifting the expandable member 160 coupled to the distal end of the catheter assembly 150 from the delivery configuration toward and/or to the deployed configuration within the blood vessel 30. In some embodiments, the method of sealing the access site 10 through the side wall 20 of the blood vessel 30 of the patient may comprise retracting the closure system 100 away from and/or relative to the blood vessel 30 to engage the expandable member 160 with the interior surface 22 of the side wall 20 of the blood vessel 30, as seen in FIG. 6.

In some embodiments, the closure system 100 may comprise a sealing shaft assembly 170 extending distally from the device handle 120, as seen in the partial cutaway detail view of FIG. 3. The sealing shaft assembly 170 may be operatively connected to and/or in electronic communication with a source of RF energy 104. In some embodiments, the source of RF energy 104 may include and/or may be an RF generator. Other configurations are also contemplated. In at least some embodiments, the sealing shaft assembly 170 may be configured to apply RF energy to the side wall 20 of the blood vessel 30 of the patient. In some embodiments, the sealing shaft assembly 170 may comprise a first shaft portion 172 fixedly secured to the device handle 120 and/or the main handle 122, and a second shaft portion 174 movably coupled to the first shaft portion 172. Some suitable but non-limiting materials for the sealing shaft assembly 170, the first shaft portion 172, and/or the second shaft portion 174, etc., including but not limited to polymeric materials, metallic materials, and/or composite materials, are described below. Other configurations and/or materials are also contemplated.

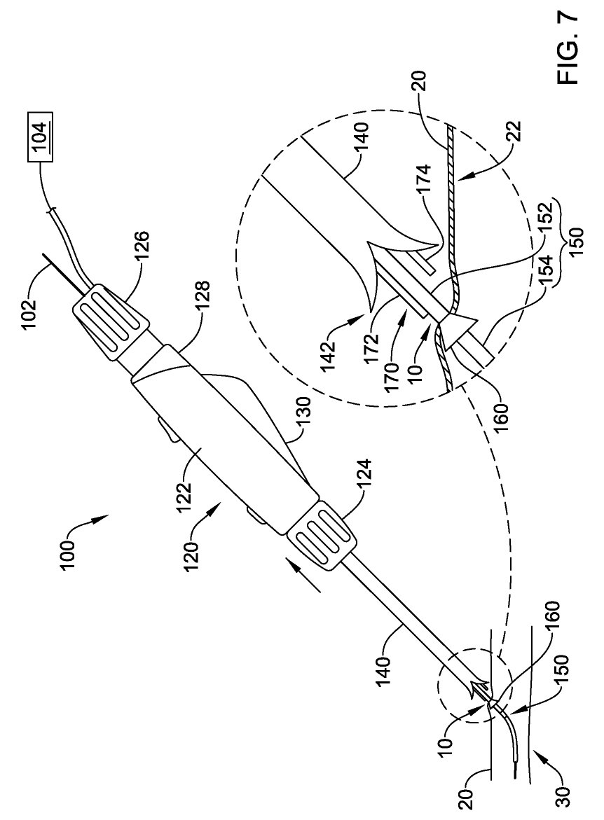

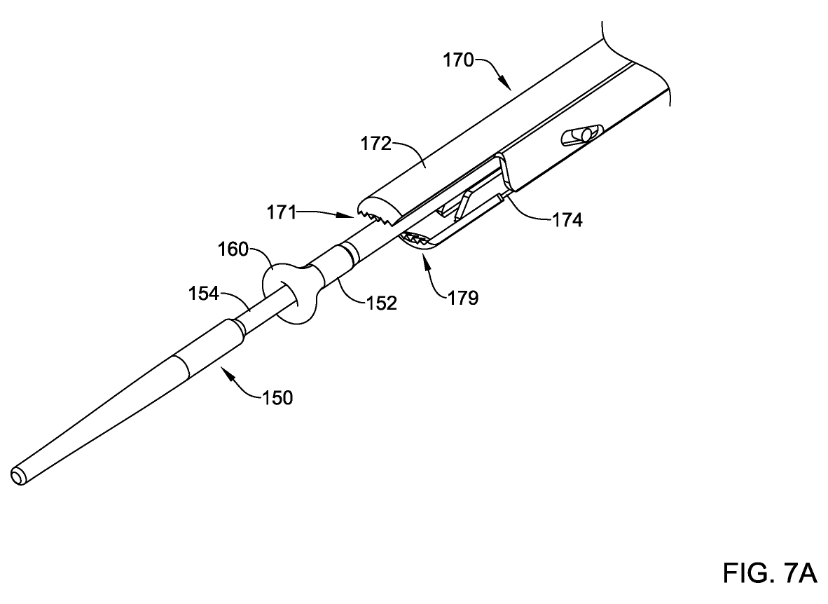

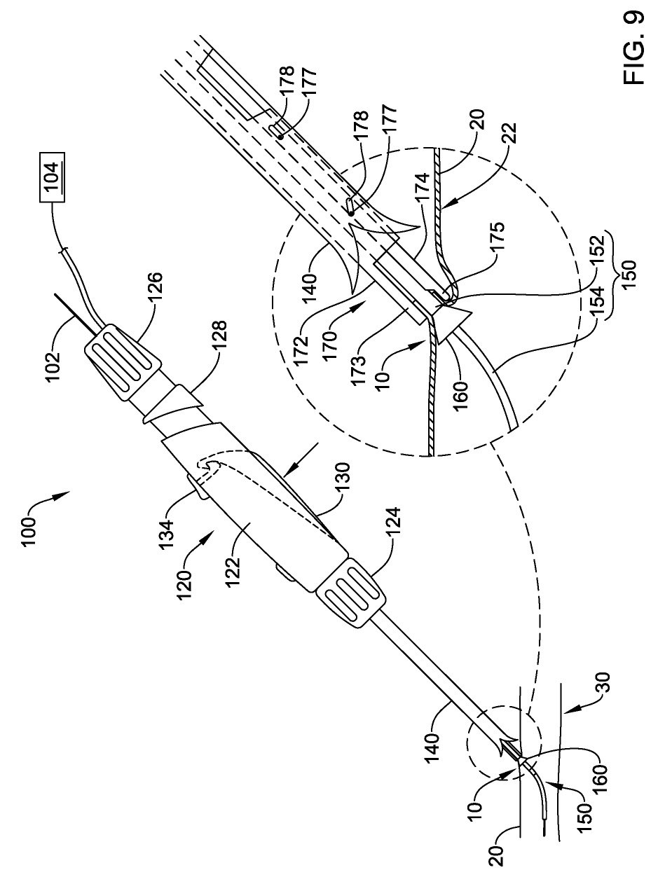

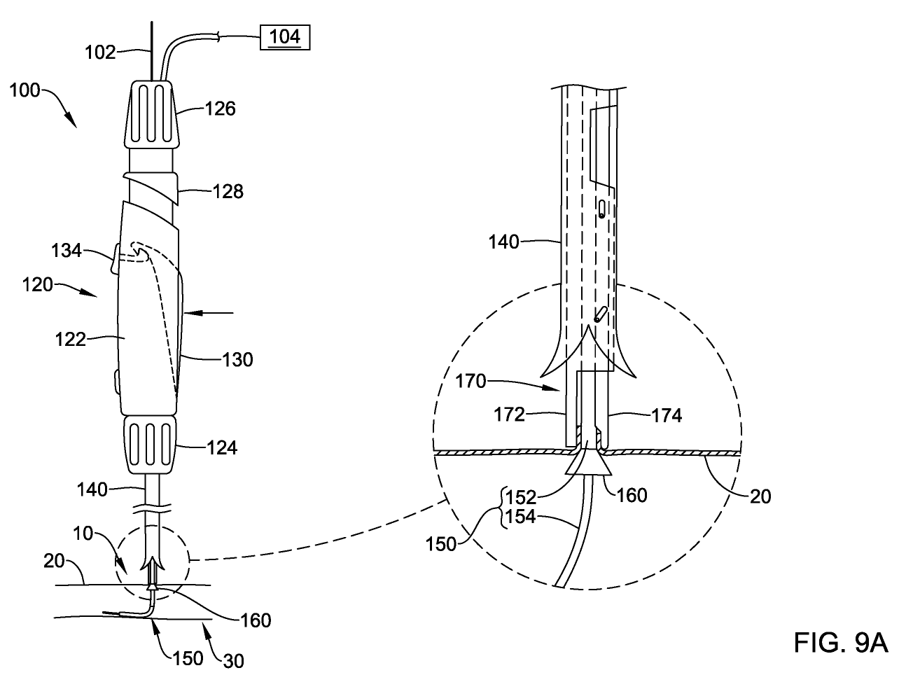

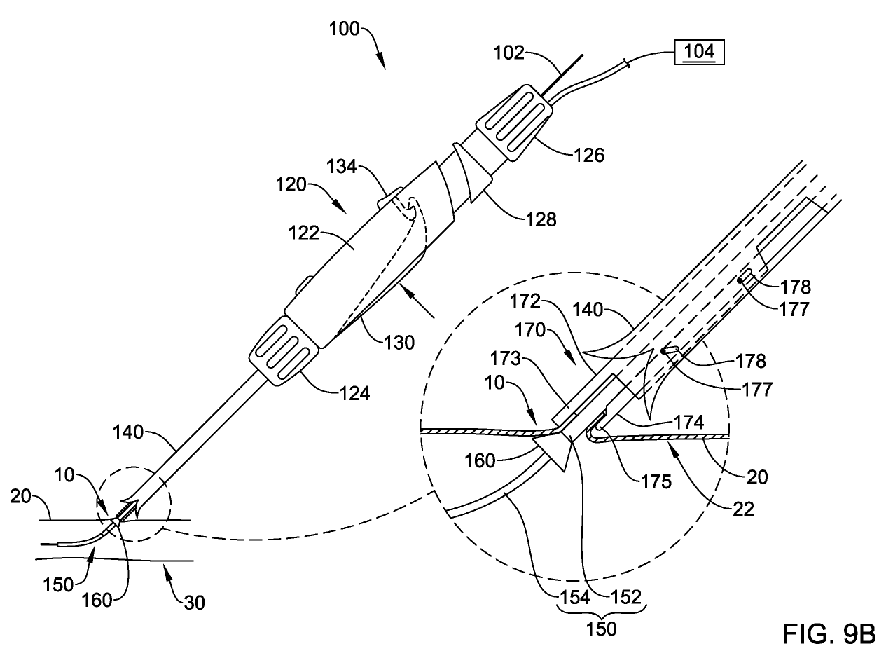

In some embodiments, the second shaft portion 174 may be operatively coupled to the actuator 130 of the device handle 120. In some embodiments, the actuator 130 may be configured to move the second shaft portion 174 relative to the first shaft portion 172 between an open configuration (e.g., FIG. 7) and a closed configuration (e.g., FIGS. 9, 9A, 9B).

In some embodiments, a distal end of the second shaft portion 174 may be axially offset from a distal end of the first shaft portion 172 in the open configuration. In some embodiments, operation and/or actuation of the actuator 130 may be configured to shift the second shaft portion 174 axially and laterally relative to the first shaft portion 172 when shifting the sealing shaft assembly 170 between the open configuration and the closed configuration. In some embodiments, the sealing shaft assembly 170 may comprise a linkage 176 (e.g., FIG. 3) configured to cooperate with the actuator 130 to move the second shaft portion 174 axially and/or laterally relative to the first shaft portion 172. In some embodiments, the linkage 176 may comprise at least one pin 177 or at least one extension element extending from the second shaft portion 174 and at least one slot 178 formed in the first shaft portion 172 configured to slidably receive the at least one pin 177 or the at least one extension element, as seen in FIG. 3. Other configurations are also contemplated.

In some embodiments, the catheter assembly 150 may extend within the sealing shaft assembly 170. In some embodiments, the catheter assembly 150 may be slidably disposed between the first shaft portion 172 and the second shaft portion 174 of the sealing shaft assembly 170. In some embodiments, the catheter assembly 150 may be axially translatable within and/or relative to the sealing shaft assembly 170.

In some embodiments, the outer sheath 140 may be fixedly attached and/or fixedly secured to the distal knob 124. In some embodiments, the catheter assembly 150 may extend distally from the device handle 120 within the outer sheath 140. In some embodiments, the sealing shaft assembly 170 may extend distally from the device handle 120 within the outer sheath 140. In some embodiments, in a delivery arrangement of the outer sheath 140, the distal opening 142 of the outer sheath 140 may have a diameter less than an outermost radial extent of the sealing shaft assembly 170 and/or greater than an outermost radial extent of the catheter assembly 150.

In some embodiments, the distal knob 124 may be configured to axially translate the outer sheath 140 relative to the sealing shaft assembly 170 to expose a distal portion of the sealing shaft assembly 170 at and/or adjacent the access site 10. For example, the distal knob 124 may be axially moved and/or axially translated relative to the main handle 122 of the device handle 120 to axially move and/or axially translate the outer sheath 140 relative to the sealing shaft assembly 170. Other configurations and/or movements of the distal knob 124 to axially move and/or axially translate the outer sheath 140 relative to the sealing shaft assembly 170 are also contemplated.

In some embodiments, the distal opening 142 of the outer sheath 140 may be configured to expand and/or a distal portion of the outer sheath 140 may be configured to tear open when the outer sheath 140 is axially moved and/or axially translated in a proximal direction relative to the sealing shaft assembly 170, thereby exposing the distal portion of the sealing shaft assembly 170, as seen in FIG. 7. In some embodiments, the method of sealing the access site 10 through the side wall 20 of the blood vessel 30 of the patient may comprise exposing the sealing shaft assembly 170 operatively coupled to the source of RF energy 104 at and/or adjacent the access site 10. In some embodiments, exposing the sealing shaft assembly 170 may comprise axially moving and/or axially translating the distal knob 124 in the proximal direction to axially move and/or axially translate the outer sheath 140 in the proximal direction relative to the sealing shaft assembly 170. In some embodiments, exposing the sealing shaft assembly 170 may comprise positioning the distal portion of the sealing shaft assembly 170 outside of and/or distal of the outer sheath 140.

FIG. 7A is a partial detail view of selected elements shown in FIG. 7. For clarity, some elements of the closure system 100 (e.g., FIG. 7) are not shown or are shown schematically. In some embodiments, the first shaft portion 172 of the sealing shaft assembly 170 may comprise at least one first gripper element 171. In some embodiments, the at least one first gripper element 171 may be disposed at and/or proximate the distal end of the first shaft portion 172. In some embodiments, the at least one first gripper element 171 may comprise at least one tooth, at least one hook, at least one traction member, at least one tissue-engaging element, etc. In one non-limiting example, the at least one first gripper element 171 may comprise a plurality of teeth. Other configurations are also contemplated. In some embodiments, the second shaft portion 174 of the sealing shaft assembly 170 may comprise at least one second gripper element 179. In some embodiments, the at least one second gripper element 179 may be disposed at and/or proximate the distal end of the second shaft portion 174. In some embodiments, the at least one second gripper element 179 may comprise at least one tooth, at least one hook, at least one traction member, at least one tissue-engaging element, etc. In one non-limiting example, the at least one second gripper element 179 may comprise a plurality of teeth. Other configurations are also contemplated. In some embodiments, the at least one first gripper element 171 and the at least one second gripper element 179 may be configured the grasp and hold a portion of the side wall 20 of the blood vessel 30 surrounding the access site 10.

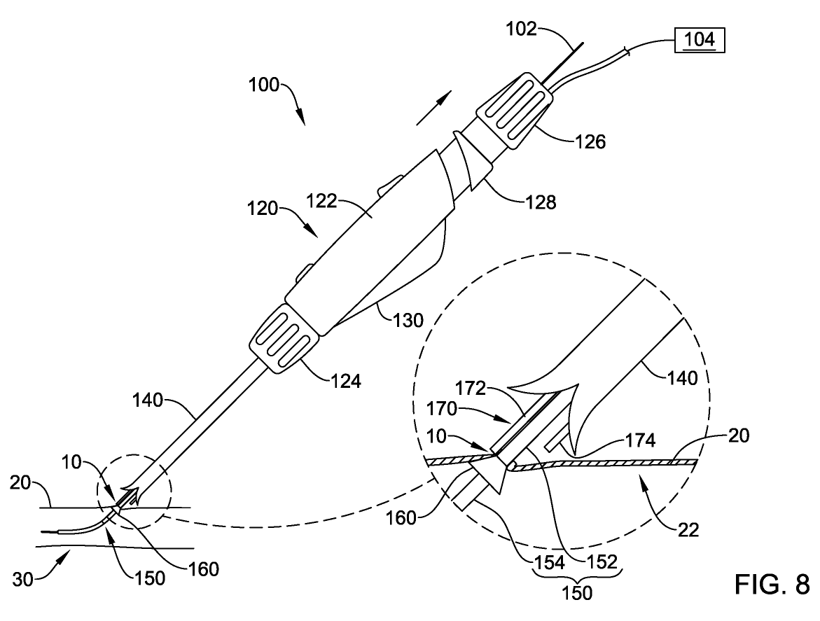

In some embodiments, the method of sealing the access site 10 through the side wall 20 of the blood vessel 30 of the patient may comprise everting and/or prolapsing a portion of the side wall 20 of the blood vessel 30 surrounding the access site 10 radially outward, as seen in FIG. 8. In some embodiments, everting and/or prolapsing the portion of the side wall 20 of the blood vessel 30 surrounding the access site 10 radially outward may comprise axially moving and/or translating the catheter assembly 150 and/or the expandable member 160 in the proximal direction relative to the sealing shaft assembly 170. In some embodiments, everting and/or prolapsing the portion of the side wall 20 of the blood vessel 30 surrounding the access site 10 radially outward and/or axially moving and/or translating the catheter assembly 150 and/or the expandable member 160 in the proximal direction relative to the sealing shaft assembly 170 may comprise axially moving and/or axially translating the proximal slide 128 in the proximal direction relative to the main handle 122, as seen in FIG. 8. In some embodiments, the proximal slide 128 may be fixedly secured to a proximal end of the catheter assembly 150 and/or a proximal end of the inner shaft member 154. In some embodiments, axially moving and/or axially translating the proximal slide 128 in the proximal direction may be configured to also move the proximal knob 126 by a same amount. In some embodiments, the proximal knob 126 may be movable completely independently of the proximal slide 128. In some embodiments, movement of the proximal slide 128 commensurately moves the proximal knob 126. Other configurations, including combinations thereof, are also contemplated.

In some embodiments, everting and/or prolapsing the portion of the side wall 20 of the blood vessel 30 surrounding the access site 10 radially outward may comprise pulling and/or drawing the portion of the side wall 20 of the blood vessel 30 surrounding the access site 10 into contact with the sealing shaft assembly 170, the first shaft portion 172, and/or the second shaft portion 174, as seen in FIG. 8. In some embodiments, everting and/or prolapsing the portion of the side wall 20 of the blood vessel 30 surrounding the access site 10 radially outward may comprise pulling and/or drawing the portion of the side wall 20 of the blood vessel 30 surrounding the access site 10 into contact with the sealing shaft assembly 170, the first shaft portion 172, and/or the second shaft portion 174 by retracting the expandable member 160 in the proximal direction via axially moving and/or axially translating the proximal slide 128 and/or the proximal knob 126 relative to the main handle 122.

In some embodiments, the method of sealing the access site 10 through the side wall 20 of the blood vessel 30 of the patient may comprise actuating the sealing shaft assembly 170 to grasp the portion of the side wall 20 of the blood vessel 30 surrounding the access site 10 that is or has been everted or prolapsed around the catheter assembly 150, as seen in FIG. 9.

In some embodiments, actuating the sealing shaft assembly 170 may comprise operating and/or actuating the actuator 130. In one non-limiting example, the actuator 130 may be squeezed against and/or into the main handle 122. Other configurations are also contemplated. In some embodiments, operating and/or actuating the actuator 130 may engage the actuator 130 with the actuator release button 134 when the sealing shaft assembly 170 is in the closed configuration to hold and/or maintain the sealing shaft assembly 170 in the closed configuration. In some embodiments, in the closed configuration, the first shaft portion 172 and the second shaft portion 174 may cooperate to exert a compressive force against the portion of the side wall 20 of the blood vessel 30 surrounding the access site 10 that is or has been everted or prolapsed, and/or the catheter assembly 150 disposed therein and/or extending therethrough. In some embodiments, in the closed configuration, the first shaft portion 172 and the second shaft portion 174 may cooperate to exert a compressive force against the portion of the side wall 20 of the blood vessel 30 surrounding the access site 10 that is or has been everted or prolapsed, and/or the outer tubular member 152 of the catheter assembly 150 disposed therein and/or extending therethrough.

In some embodiments, operating and/or actuating the actuator 130 may shift the second shaft portion 174 of the sealing shaft assembly 170 axially and/or laterally relative to the first shaft portion 172 of the sealing shaft assembly 170 when shifting the sealing shaft assembly 170 between the open configuration and the closed configuration. In some embodiments, operating and/or actuating the actuator 130 may shift the second shaft portion 174 of the sealing shaft assembly 170 distally and/or laterally inward relative to the first shaft portion 172 of the sealing shaft assembly 170 when shifting the sealing shaft assembly 170 from the open configuration to the closed configuration. In some embodiments, operating and/or actuating the actuator 130 may shift the second shaft portion 174 of the sealing shaft assembly 170 proximally and/or laterally outward relative to the first shaft portion 172 of the sealing shaft assembly 170 when shifting the sealing shaft assembly 170 from the closed configuration to the open configuration. In some embodiments, the linkage 176 may be configured to convert axial movement (and/or a portion of the axial movement) of the second shaft portion 174 relative to the first shaft portion 172 into lateral movement of the second shaft portion 174 relative to the first shaft portion 172. Accordingly, the linkage 176 may cooperate with the actuator 130 to facilitate the axial and/or lateral movement of second shaft portion 174 relative to the first shaft portion 172 required to shift the sealing shaft assembly 170 between the open configuration and the closed configuration.

In some embodiments, the distal end of the first shaft portion 172 may axially align with the distal end of the second shaft portion 174 in the closed configuration, as seen in FIGS. 9 and 9A. In some embodiments, the closure system 100 and/or the sealing shaft assembly 170 may be moved and/or shifted toward a right-angle orientation relative to the side wall 20 of the blood vessel 30, as seen in FIG. 9A. In some embodiments, the right-angle orientation may permit the access site 10 to be closed and/or sealed generally parallel to the lumen of the blood vessel 30, thereby reducing vascular turbulence and/or smoothing blood flow within the lumen of the blood vessel 30, which may reduce undesirable thrombus formation. In some embodiments, the distal end of the first shaft portion 172 may be axially offset relative to the distal end of the second shaft portion 174 in the closed configuration, as seen in FIG. 9B. In some embodiments, the closure system 100 and/or the sealing shaft assembly 170 may be held in and/or moved toward an oblique-angle orientation relative to the side wall 20 of the blood vessel 30, as seen in FIG. 9B. In some embodiments, the oblique-angle orientation may permit the access site 10 to be closed and/or sealed generally parallel to the lumen of the blood vessel 30, thereby reducing vascular turbulence and/or smoothing blood flow within the lumen of the blood vessel 30, which may reduce undesirable thrombus formation.

In some embodiments, the closure system 100 and/or the sealing shaft assembly 170 may be sized and/or configured to grasp the side wall 20 of the blood vessel 30 at the access site 10, and/or the portion of the side wall 20 of the blood vessel 30 surrounding the access site 10 that is or has been everted or prolapsed. In some embodiments, the closure system 100 and/or the sealing shaft assembly 170 may be scalable for different sizes of access sites. For example, the closure system 100 and/or the sealing shaft assembly 170 may be made in several different sizes to facilitate use with access sites having different ranges of size/diameter.

In at least some embodiments, the portion of the side wall 20 of the blood vessel 30 surrounding the access site 10 that is or has been everted or prolapsed around the catheter assembly 150 may be grasped and/or held, at least in part, by the at least one first gripper element 171 (e.g., FIG. 7A) and the at least one second gripper element 179 (e.g., FIG. 7A) of the sealing shaft assembly 170.

In some embodiments, the sealing shaft assembly 170 may comprise bipolar electrodes configured to deliver and/or transmit RF energy to adjacent tissue(s). In some embodiments, a distal end region of the first shaft portion 172 may comprise a first polarity electrode 173 (e.g., FIG. 9). In some embodiments, a distal end region of the second shaft portion 174 may comprise a second polarity electrode 175 (e.g., FIG. 9). In some embodiments, the first polarity electrode 173 and the second polarity electrode 175 may form and/or define a current path therebetween when the source of RF energy 104 is activated. It is believed that a bipolar system may provide improved accuracy of tissue heating and/or tissue fusion over a monopolar system, such that sealing of the access site 10 will be improved with less (or no) collateral tissue damage.

In some embodiments, the sealing shaft assembly 170, the catheter assembly 150, the outer tubular member 152, and/or the outer sheath 140 may comprise an insulating material (or a plurality of insulating materials) and/or a coating (or a plurality of coatings) configured to electrically isolate the bipolar electrodes (e.g., the first polarity electrode 173 and the second polarity electrode 175) from other elements and/or structures of the closure system 100. In some embodiments, the insulating material(s) and/or the coating(s) may be formed from a polymeric material, a ceramic material, etc. Some suitable but non-limiting examples of materials for the insulating material are discussed below.

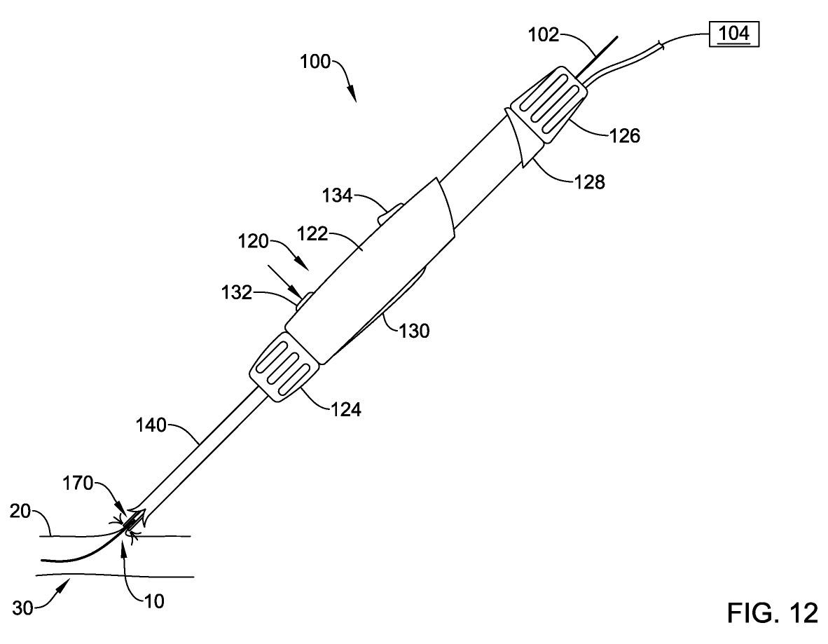

In some embodiments, the method of sealing the access site 10 through the side wall 20 of the blood vessel 30 of the patient may comprise applying RF energy to the side wall 20 of the blood vessel 30 at the access site 10 with the sealing shaft assembly 170, as seen in FIG. 12. In some embodiments, the method of sealing the access site 10 through the side wall 20 of the blood vessel 30 of the patient may comprise applying RF energy to the portion of the side wall 20 of the blood vessel 30 surrounding the access site 10 that is or has been everted or prolapsed with the sealing shaft assembly 170. In some embodiments, the method of sealing the access site 10 through the side wall 20 of the blood vessel 30 of the patient may comprise applying RF energy to the portion of the side wall 20 of the blood vessel 30 surrounding the access site 10 that is or has been everted or prolapsed with the sealing shaft assembly 170 while applying the compressive force (e.g., pressure) with the sealing shaft assembly 170 (e.g., the first shaft portion 172 and the second shaft portion 174) to the portion of the side wall 20 of the blood vessel 30 surrounding the access site 10 that is or has been everted or prolapsed. In at least some embodiments, a combination of the compressive force (e.g., pressure) and the RF energy applied to the portion of the side wall 20 of the blood vessel 30 surrounding the access site 10 that is or has been everted or prolapsed may close and/or seal the access site 10.

In some embodiments, the RF actuation button 132 may be configured to activate the source of RF energy 104. In some embodiments, the method of sealing the access site 10 through the side wall 20 of the blood vessel 30 of the patient may comprise operating and/or activating the RF actuation button 132 to apply RF energy to the side wall 20 of the blood vessel 30 at the access site 10, and/or the portion of the side wall 20 of the blood vessel 30 surrounding the access site 10 that is or has been everted or prolapsed, with the sealing shaft assembly 170. In some embodiments, the RF actuation button 132 may be configured to be pressed by a finger or a thumb of the user. In some embodiments, the RF actuation button 132 may be touch sensitive. In some embodiments, the RF actuation button 132 may be a slider or a lever. In some embodiments, the RF actuation button 132 may be disposed remotely from the device handle 120. In some such embodiments, the device handle 120 may be devoid of the actuator release button 134. In some embodiments, the RF actuation button 132 may be a foot switch. In some embodiments, the RF actuation button 132 may be disposed on the source of RF energy 104 (e.g., on the RF generator).

Other configurations are also contemplated.

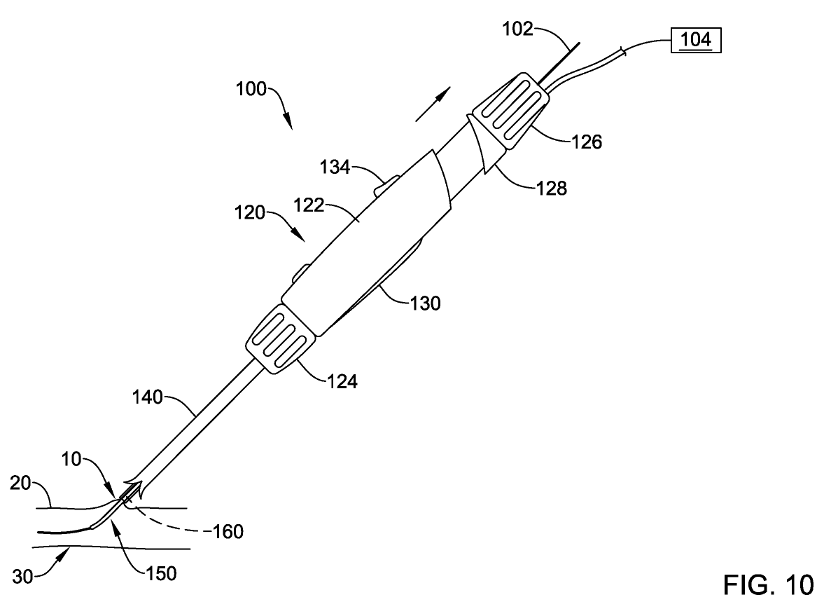

Returning to FIG. 10, in some embodiments, the method of sealing the access site 10 through the side wall 20 of the blood vessel 30 of the patient may comprise, prior to applying RF energy to the side wall 20 of the blood vessel 30, and/or the portion of the side wall 20 of the blood vessel 30 surrounding the access site 10 that is or has been everted or prolapsed, and while grasping the portion of the side wall 20 of the blood vessel 30 surrounding the access site 10 that is or has been everted or prolapsed with the sealing shaft assembly 170, retracting the expandable member 160 within the catheter assembly 150. In some embodiments, retracting the expandable member 160 within the catheter assembly 150 may comprise axially moving and/or axially translating the proximal slide 128 in the proximal direction relative to the main handle 122. In some embodiments, retracting the expandable member 160 within the catheter assembly 150 may comprise axially moving and/or axially translating the proximal slide 128 in the proximal direction relative to the main handle 122 an additional amount or distance beyond what the proximal slide 128 was moved previously. In some embodiments, retracting the expandable member 160 within the catheter assembly 150 may comprise axially moving and/or axially translating the proximal slide 128 into engagement with and/or into contact with the proximal knob 126. Other configurations are also contemplated. In some embodiments, the at least one first gripper element 171 (e.g., FIG. 7A) and the at least one second gripper element 179 (e.g., FIG. 7A) of the sealing shaft assembly 170 may prevent the portion of the side wall 20 of the blood vessel 30 surrounding the access site 10 that is or has been everted or prolapsed from being released by and/or slipping out of the sealing shaft assembly 170 in the closed configuration as the expandable member 160 is retracted within the catheter assembly 150.

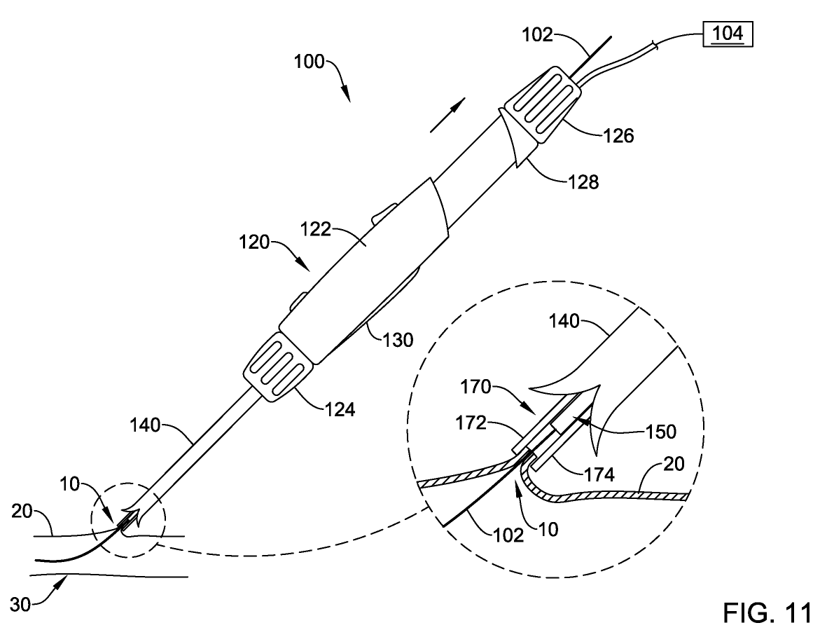

In some embodiments, the method of sealing the access site 10 through the side wall 20 of the blood vessel 30 of the patient may comprise, prior to applying RF energy to the side wall 20 of the blood vessel 30, and/or the portion of the side wall 20 of the blood vessel 30 surrounding the access site 10 that is or has been everted or prolapsed, after retracting the expandable member 160 within the catheter assembly 150, and while grasping the portion of the side wall 20 of the blood vessel 30 surrounding the access site 10 that is or has been everted or prolapsed with the sealing shaft assembly 170, retracting and/or withdrawing the catheter assembly 150 from the blood vessel 30 through the access site 10 in the side wall 20 of the blood vessel 30 to a retracted position within the closure system 100 and/or within the sealing shaft assembly 170, as seen in FIG. 11. In some embodiments, the catheter assembly 150 may be axially movable and/or axially translatable relative to the sealing shaft assembly 170 when the sealing shaft assembly 170 is in the closed configuration. In some embodiments, the sealing shaft assembly 170 may be biased toward the closed configuration such that as the catheter assembly 150 is withdrawn through the sealing shaft assembly and/or between the first shaft portion 172 and the second shaft portion 174 in the closed configuration, the second shaft portion 174 may shift toward the first shaft portion 172 to close any gap within the access site 10 left by the removal of the catheter assembly 150, thereby maintaining a grasp on the side wall 20 of the blood vessel 30, and/or the portion of the side wall 20 of the blood vessel 30 surrounding the access site 10 that is or has been everted or prolapsed. In some embodiments, the at least one first gripper element 171 (e.g., FIG. 7A) and the at least one second gripper element 179 (e.g., FIG. 7A) of the sealing shaft assembly 170 may prevent the portion of the side wall 20 of the blood vessel 30 surrounding the access site 10 that is or has been everted or prolapsed from being released be and/or slipping out of the sealing shaft assembly 170 in the closed configuration as the catheter assembly 150 is retracted and/or withdrawn from the blood vessel 30 through the access site 10 in the side wall 20 of the blood vessel 30 to the retracted position within the closure system 100 and/or within the sealing shaft assembly 170.

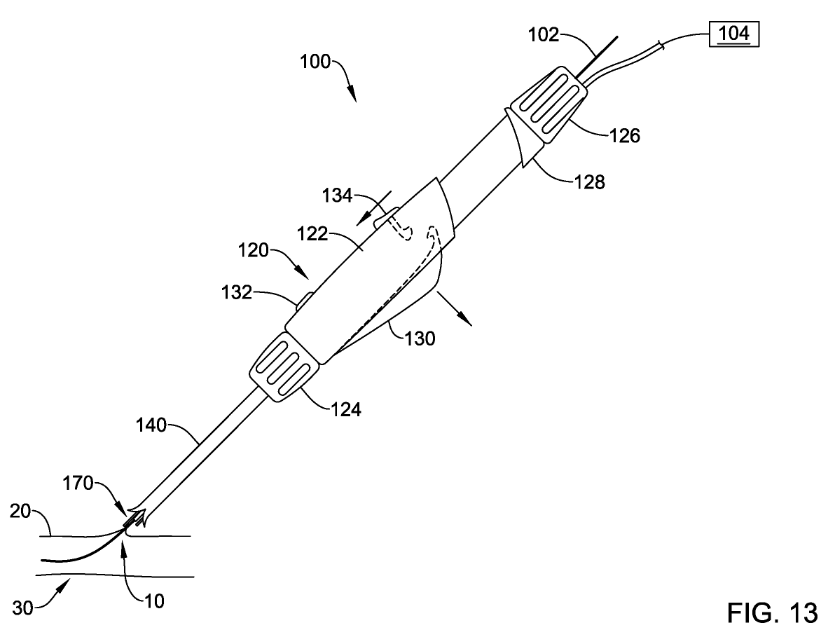

In some embodiments, the method of sealing the access site 10 through the side wall 20 of the blood vessel 30 of the patient may comprise, after applying RF energy to the side wall 20 of the blood vessel 30, and/or the portion of the side wall 20 of the blood vessel 30 surrounding the access site 10 that is or has been everted or prolapsed, as seen in FIG. 12, actuating the sealing shaft assembly 170 to release the side wall 20 of the blood vessel 30 and/or the portion of the side wall 20 of the blood vessel 30 surrounding the access site 10 that is or has been everted or prolapsed, as seen in FIG. 13. In some embodiments, actuating the sealing shaft assembly 170 to release the side wall 20 of the blood vessel 30 and/or the portion of the side wall 20 of the blood vessel 30 surrounding the access site 10 that is or has been everted or prolapsed comprises activating the actuator release button 134, thereby permitting the actuator 130 to return to an unactuated position corresponding to the open configuration of the sealing shaft assembly 170. In some embodiments, the actuator 130 may be biased toward and/or to the unactuated position. In some embodiments, the actuator 130 may be self-biased toward and/or to the unactuated position. In some embodiments, the sealing shaft assembly 170 may be biased toward and/or to the open configuration. In some embodiments, the sealing shaft assembly 170 may be self-biased toward and/or to the open configuration. In some embodiments, after releasing the side wall 20 of the blood vessel 30 and/or the portion of the side wall 20 of the blood vessel 30 surrounding the access site 10 that is or has been everted or prolapsed, the access site 10 may be checked (e.g., visually or via other means) for leakage. In some embodiments, if the access site 10 leaks blood, the above steps may be repeated. In some embodiments, if the access site 10 does not leak blood, the user may move on toward completion of the procedure.

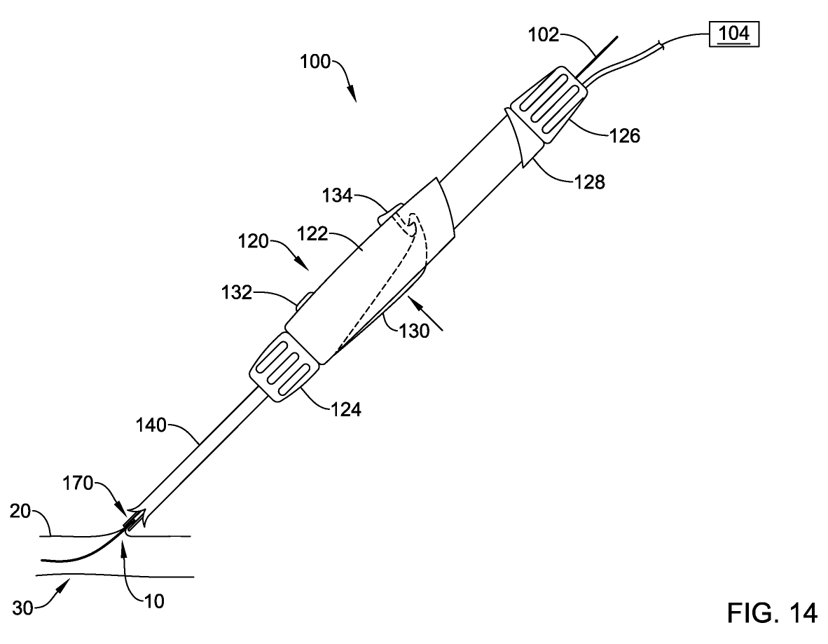

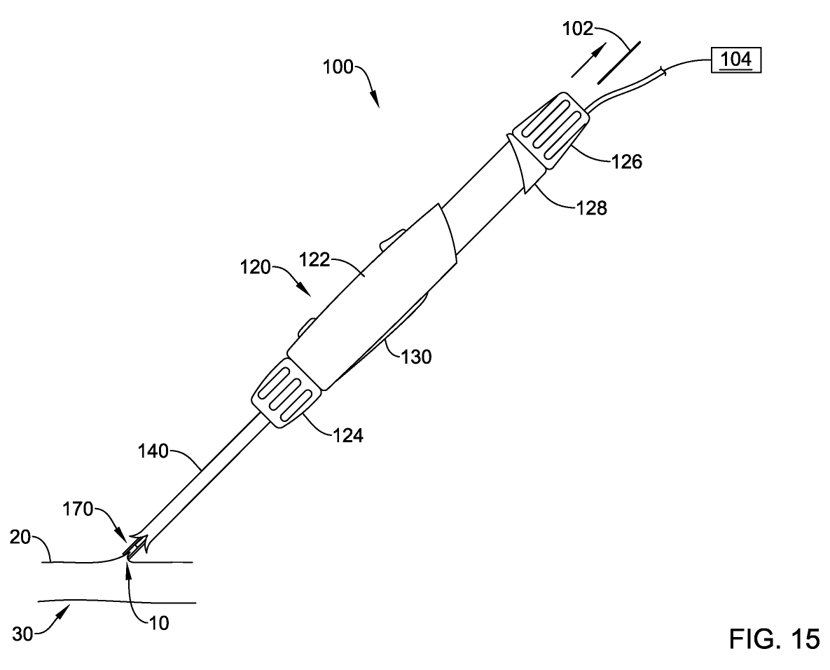

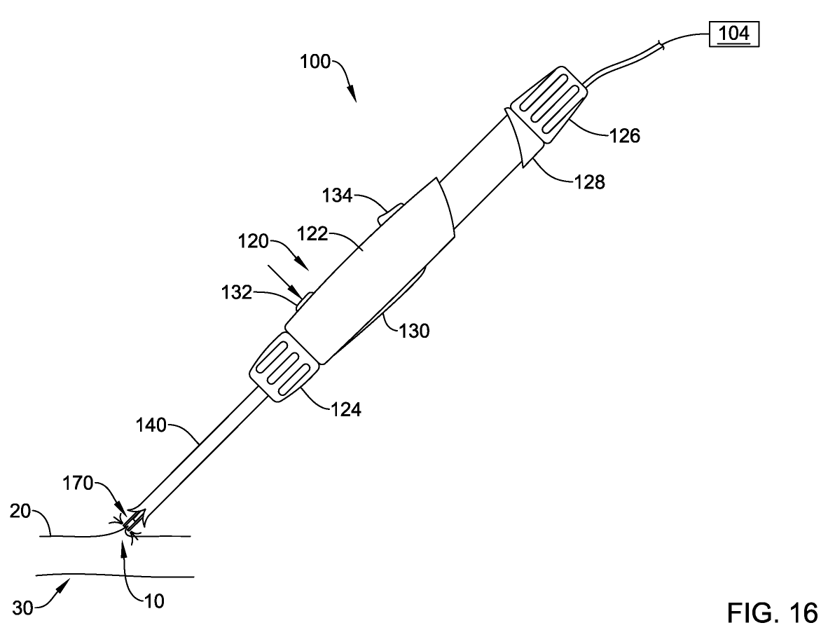

In some embodiments, the method of sealing the access site 10 through the side wall 20 of the blood vessel 30 of the patient may comprise actuating the sealing shaft assembly 170 (for a second time) to re-grasp the portion of the side wall 20 of the blood vessel 30 surrounding the access site 10 that is or has been everted or prolapsed around the catheter assembly 150, as seen in FIG. 14. In some embodiments, the method of sealing the access site 10 through the side wall 20 of the blood vessel 30 of the patient may comprise withdrawing the guidewire 102 from the access site 10 into and/or through the guidewire lumen of the catheter assembly 150, as seen in FIG. 15. In some embodiments, the method of sealing the access site 10 through the side wall 20 of the blood vessel 30 of the patient may comprise, thereafter, applying RF energy to the side wall 20 of the blood vessel 30 at the access site 10, and/or to the portion of the side wall 20 of the blood vessel 30 surrounding the access site 10 that is or has been everted or prolapsed, with the sealing shaft assembly 170, as seen in FIG. 16. A second application of RF energy may seal any remaining opening at the access site 10 after the guidewire 102 has been removed. In some embodiments, a second application of RF energy, and any associated steps/actions, may be unnecessary and/or may be omitted.

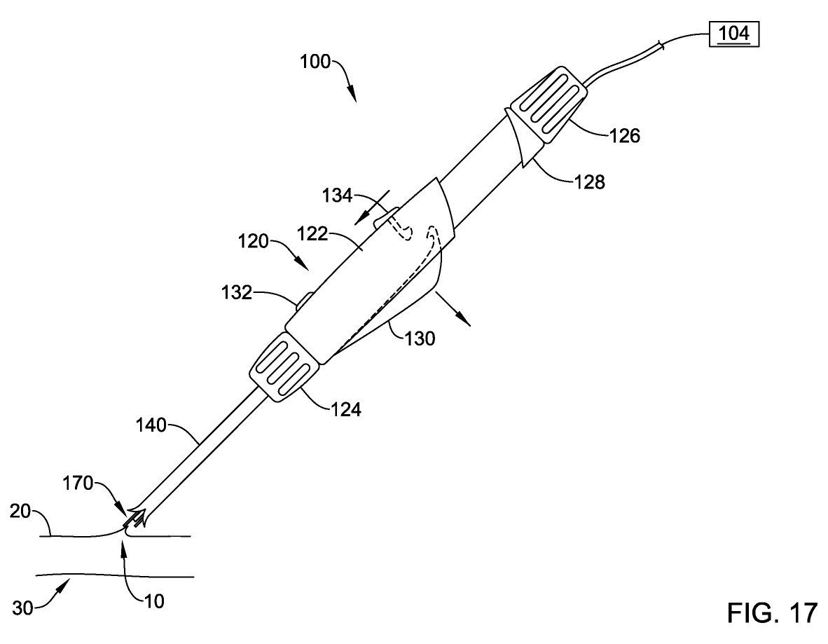

In some embodiments, the method of sealing the access site 10 through the side wall 20 of the blood vessel 30 of the patient may comprise, after applying RF energy to the side wall 20 of the blood vessel 30, and/or the portion of the side wall 20 of the blood vessel 30 surrounding the access site 10 that is or has been everted or prolapsed, for a second time, actuating the sealing shaft assembly 170 to release the side wall 20 of the blood vessel 30 and/or the portion of the side wall 20 of the blood vessel 30 surrounding the access site 10 that is or has been everted or prolapsed, as seen in FIG. 17. In some embodiments, actuating the sealing shaft assembly 170 to release the side wall 20 of the blood vessel 30 and/or the portion of the side wall 20 of the blood vessel 30 surrounding the access site 10 that is or has been everted or prolapsed comprises activating the actuator release button 134, thereby permitting the actuator 130 to return to an unactuated position corresponding to the open configuration of the sealing shaft assembly 170.

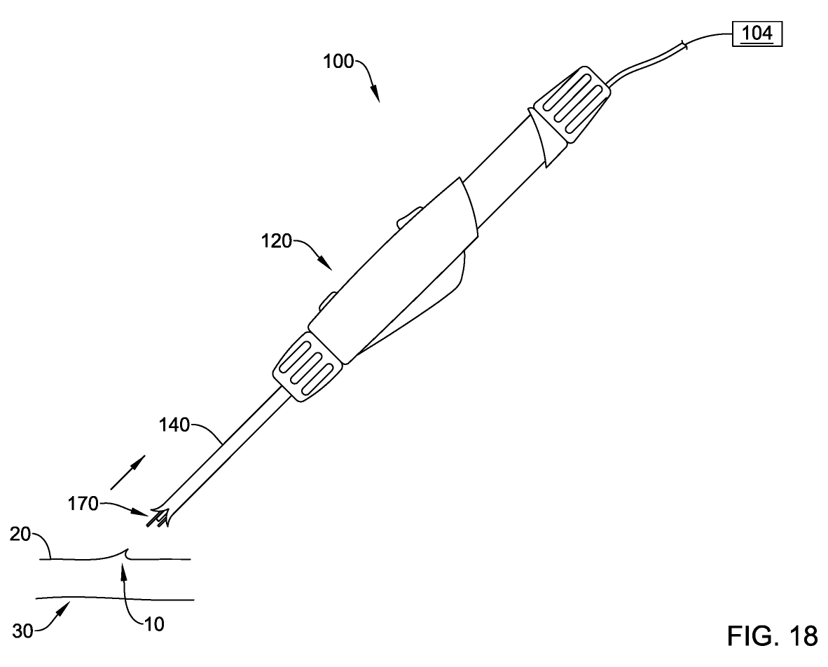

In some embodiments, the method of sealing the access site 10 through the side wall 20 of the blood vessel 30 of the patient may comprise, after sealing the access site 10, removing the closure system 100 from the access site 10 and/or the patient, as seen in FIG. 18. In some embodiments, the closure system 100 may be devoid of any structure configured to be left behind within the blood vessel 30 (e.g., such as a collagen plug or a structure pulled against the interior surface 22 of the side wall 20 of the blood vessel 30) after the sealing shaft assembly 170 applies RF energy to the side wall 20 of the blood vessel 30, and/or the portion of the side wall 20 of the blood vessel 30 surrounding the access site 10 that is or has been everted or prolapsed. In some embodiments, the closure system 100 may be devoid of any structure configured to be left attached to the blood vessel 30 (e.g., such as a clip, suture, staple, etc.) after the sealing shaft assembly 170 applies RF energy to the side wall 20 of the blood vessel 30, and/or the portion of the side wall 20 of the blood vessel 30 surrounding the access site 10 that is or has been everted or prolapsed. As such, the closure system 100 may be considered as a “nothing left behind” closure device, which provides one or more advantages over prior art closure devices that do leave structure(s) behind. For example, leaving no structure(s) behind within or attached to the blood vessel 30 eliminates foreign bodies that may promote thrombus formation, that may cause infection, and/or that may delay or inhibit healing.

The materials that can be used for the various components of the closure system (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the device handle, the catheter assembly, the outer sheath, the sealing shaft assembly, the expandable member, etc. and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, acrylonitrile butadiene styrene (ABS), epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP).

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or components thereof may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment in other embodiments. The scope of the disclosure is, of course, defined in the language in which the appended claims are expressed.

What is claimed is:

1. A closure system for sealing an access site through a side wall of a blood vessel of a patient, comprising:
a device handle;
a catheter assembly extending distally from the device handle;
an expandable member coupled to a distal end of the catheter assembly and configured to shift between a delivery configuration and a deployed configuration, wherein the expandable member is configured to engage an interior surface of the side wall of the blood vessel in the deployed configuration; and
a sealing shaft assembly extending distally from the device handle and operatively connected to a source of RF energy, wherein the sealing shaft assembly is configured to apply RF energy to the side wall of the blood vessel at the access site.

2. The closure system of claim 1, wherein the expandable member comprises a framework including a plurality of tissue-engaging elements extending therefrom.

3. The closure system of claim 1, wherein the expandable member is self-biased toward the deployed configuration.

4. The closure system of claim 1, wherein the catheter assembly comprises an outer tubular member fixedly secured to a first end of the expandable member and an inner shaft member fixedly secured to a second end of the expandable member.

5. The closure system of claim 4, wherein the inner shaft member is axially movable relative to the outer tubular member to shift the expandable member between the delivery configuration and the deployed configuration.

6. The closure system of claim 1, wherein the catheter assembly extends within the sealing shaft assembly.

7. The closure system of claim 1, wherein the sealing shaft assembly comprises a first shaft portion fixedly secured to the device handle and a second shaft portion movably coupled to the first shaft portion, wherein the second shaft portion is operatively coupled to an actuator of the device handle.

8. The closure system of claim 7, wherein the actuator is configured to move the second shaft portion relative to the first shaft portion to shift the sealing shaft assembly between an open configuration and a closed configuration.

9. The closure system of claim 8, wherein the sealing shaft assembly comprises a linkage configured to cooperate with the actuator to move the second shaft portion axially and laterally relative to the first shaft portion.

10. The closure system of claim 8, wherein in the closed configuration, the first shaft portion and the second shaft portion cooperate to exert a compressive force against the catheter assembly.

11. The closure system of claim 10, wherein the catheter assembly is axially translatable relative to the sealing shaft assembly when the sealing shaft assembly is in the closed configuration.

12. The closure system of claim 7, wherein the first shaft portion comprises a first polarity electrode and the second shaft portion comprises a second polarity electrode.

13. A closure system for sealing an access site through a side wall of a blood vessel of a patient, comprising:
a device handle;
a catheter assembly extending distally from the device handle;
an expandable member coupled to a distal end of the catheter assembly and configured to shift between a delivery configuration and a deployed configuration, wherein the expandable member is configured to engage an interior surface of the side wall of the blood vessel in the deployed configuration; and
a sealing shaft assembly extending distally from the device handle and operatively connected to a source of RF energy, wherein the sealing shaft assembly is configured to apply RF energy to the side wall of the blood vessel at the access site;
wherein the sealing shaft assembly comprises a first shaft portion fixedly secured to the device handle and a second shaft portion movably coupled to the first shaft portion, wherein a distal end of the second shaft portion is axially offset from a distal end of the first shaft portion in an open configuration;
wherein the catheter assembly is slidably disposed between the first shaft portion and the second shaft portion.

14. The closure system of claim 13, wherein the closure system is devoid of any structure configured to be left behind within the blood vessel after the sealing shaft assembly applies RF energy to the side wall of the blood vessel.

15. The closure system of claim 13, wherein the closure system is devoid of any structure configured to be left attached to the blood vessel after the sealing shaft assembly applies RF energy to the side wall of the blood vessel.

16. A method of sealing an access site through a side wall of a blood vessel, comprising:
advancing a catheter assembly of a closure system over a guidewire into the blood vessel through the access site;
shifting an expandable member coupled to the distal end of the catheter assembly from a delivery configuration to a deployed configuration within the blood vessel;
retracting the closure system to engage the expandable member with an interior surface of the side wall of the blood vessel;
exposing a sealing shaft assembly operatively connected to a source of RF energy adjacent the access site;
actuating the sealing shaft assembly to grasp the side wall of the blood vessel around the catheter assembly; and
applying RF energy to the side wall of the blood vessel at the access site with the sealing shaft assembly.

17. The method of claim 16, further comprising:
prior to applying RF energy to the side wall of the blood vessel, and while grasping the side wall of the blood vessel with the sealing shaft assembly, retracting the expandable member within the catheter assembly.

18. The method of claim 16, further comprising:
after applying RF energy to the side wall of the blood vessel, actuating the sealing shaft assembly to release the side wall of the blood vessel.

19. The method of claim 16, wherein the sealing shaft assembly comprises a first shaft portion fixedly secured to a device handle of the closure system and a second shaft portion movably coupled to the first shaft portion, wherein the second shaft portion is operatively coupled to an actuator of the device handle.

20. The method of claim 19, wherein the sealing shaft assembly comprises a linkage configured to cooperate with the actuator to move the second shaft portion axially and laterally relative to the first shaft portion.
